(19) United States
(12) Patent Application Publication
(10) Pub. No.: US 2026/0345474 A1
(43) Pub. Date: Nov. 5, 2026

(54) TOPICAL COSMETIC COMPOSITION FOR HAIR AND SKIN CARE

(71) Applicant: Swiss EcoNaturals AG, Zug (CH)

(72) Inventors: Nicola LIONETTI, Milan (IT); Miriam DEOLA, Milan (IT); Shivi GUPTA, Cham (CH)

(73) Assignee: Swiss EcoNaturals AG, Zug (CH)

(21) Appl. No.: 19/188,046

(22) Filed: Apr. 24, 2025

(51) Int. Cl. A61K 8/9789; A61K 8/34; A61K 8/60; A61Q 5/00; A61Q 17/04; A61Q 19/00; A61Q 19/02; A61Q 19/08

(52) U.S. Cl. CPC A61K 8/9789; A61K 8/347; A61K 8/602; A61Q 5/002; A61Q 17/04; A61Q 19/007; A61Q 19/02; A61Q 19/08

(57) ABSTRACT

The present invention relates to a hair and/or skin care cosmetic composition for topical application. The composition contains bakuchiol, an extract from Centella asiatica containing madecassoside, terminoloside and asiaticoside, and an extract of Prunus amygdalus seeds.

### FIELD AND BACKGROUND OF THE INVENTION

The present invention relates to the technical field of topical cosmetic compositions for hair and skin care.

Medicinal plants have been used widely in nutraceuticals and cosmeceuticals. In such products phytochemicals from plants represent natural sources of compounds with several biological benefits.

Centella asiatica, is also known as Violette marronne (Reunion Island), as Gotu Kola or as Indian pennywort (India), or as Centella repanda (North America) and as Talapetraka (Madagascar), is a polymorphous herb. Centella asiatica belongs to the family of Umbelliferae (Apiaceae), particularly to the Hydrocotyle subfamily that grows wild in pantropical moist and shady regions at an ideal altitude of 600 to 1200 m. Centella asiatica includes three varieties called Typica, Abyssinica and Floridana. It is known and used for healing, sedative, analgesic, antidepressant, antiviral and antimicrobial properties. The biological activity is enabled by active molecules from the triterpene series such as asiaticoside (I), madecassoside (II), terminoloside (III), asiatic acid, madecassic acid and terminolic acid. They contribute to the natural defense of the plants against environmental aggression thanks to the anti-bacterial properties of the asiatic acid, madecassic acid and terminolic acid, which are obtained by hydrolysis from the heterosidic reserve form (I, II, III).

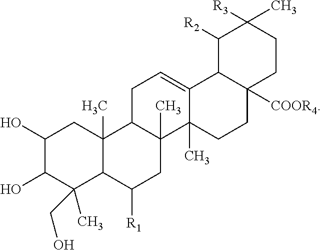

| | | R₁ | R₂ | R₃ | R₄ |
|---|---|---|---|---|---|
| I | asiaticoside | H | CH₃ | H | Glu-Glu-Rha |
| II | madecassoside | OH | CH₃ | H | Glu-Glu-Rha |
| III | terminoliside | OH | H | CH₃ | Glu-Glu-Rha |

Triterpenes are used also in cosmeceuticals and cosmetic products. They typically exhibit wound-healing, anti-wrinkle, and anti-cellulite effects by increasing the synthesis of collagen and improving the production of fibronectin in human skin fibroblasts. Specifically, asiaticoside increases the hydroxyproline content and tensile strength in wound tissue, raises the collagen Type I and III content, expedites the wound-healing process, and induces glycosaminoglycan synthesis. Madecassoside can also stimulate the production of collagen Type III. A commercially available extract from Centella asiatica is CENTEROX™ (Givaudan). CENTEROX™ appears to have hair strengthening and collagen synthesis enhancing properties and is sold for use in cosmetic composition. Centerox™ contains madecassoside, terminoloside and asiaticoside in a total amount of 70.0-90.0 wt-% as determined by HPLC, with a total amount of madecassoside and terminoloside of 50.0-70.0 wt-% as determined by HPLC and an amount of asiaticoside of 10.0-20.0 wt-% as determined by HPLC.

A need remains for further cosmetic compositions containing extracts from Centella asiatica for skin and hair care with improved properties compared to the known cosmetic products.

### BRIEF DESCRIPTION OF THE SEVERAL VIEWS OF THE DRAWINGS

FIG. 1A-C show normalized cell viability after treatment of human keratinocytes cells with A) SYTHENOL A, B) SWEET ALMOND and C) CENTEROX, compared to the non-treated control (C). Data are presented as mean±standard error of the median (SEM). Statistical significance is depicted as **** for p<0.0001, ** for p<0.01 and * for p<0.05

FIG. 2A-C show UVA-induced ROS levels normalized to cell viability data (MTT) for A) SYTHENOL A, B) SWEET ALMOND and C) CENTEROX. No statistically significant difference was found between vitamin C (Vit C) used as positive control and CENTEROX. Data are presented as mean±standard error of the median (SEM). Statistical significance is depicted as **** for p<0.0001, ** for p<0.001, ** for p<0.01 and * for p<0.05.

FIG. 3A-B show normalized cell viability after treatment of human keratinocytes cells with A) 1^{st} composition and B) 2^{nd} composition, compared to the non-treated control (C). Data are presented as mean±standard error of the median (SEM).

FIG. 4A-B show UVA-induced ROS levels normalized to cell viability data (MTT) for A) 1^{st} composition, and B) 2^{nd} composition. No statistically significant difference was found between vitamin C (Vit. C) used as positive control and the 1^{st} composition when used at 1%. Data are presented as mean±standard error of the median (SEM). Statistical significance is depicted as **** for p<0.0001, * for p<0.001, ** for p<0.01 and * for p<0.05.

### DESCRIPTION OF SPECIFIC EMBODIMENTS OF THE INVENTION

It is an object of the present to address the need for an improved cosmetic composition containing extracts from Centella asiatica for skin and hair care. The object is achieved by the cosmetic composition according to claim 1. Preferred embodiments are disclosed in the specification and the dependent claims.

The present invention will be described in more detail below.

Where the present description refers to “preferred” embodiments/features, combinations of these “preferred” embodiments/features are also deemed to be disclosed as long as the specific combination of the “preferred” embodiments/features is technically meaningful.

Unless otherwise stated, the following definitions shall apply in this specification:

As used herein, the term “a”, “an”, “the” and similar terms used in the context of the present invention (especially in the context of the claims) are to be construed to cover both the singular and plural unless otherwise indicated herein or clearly contradicted by the context.

As used herein, the term “and/or” means that either all or only one of the elements of said group may be present. For example, “A and/or B” means “only A, or only B, or both A and B”. In the case of “only A”, the term also covers the possibility that B is absent, i.e. “only A, but not B”.

As used herein, the terms “including”, “containing” and “comprising” are used herein in their open-ended, non-limiting sense. It is understood that the various embodiments, preferences and ranges may be combined at will. Thus, for instance a solution comprising a compound A may include other compounds besides A. However, the term “comprising” also covers, as a particular embodiment thereof, the more restrictive meanings of “consisting essentially of” and “consisting of”, so that for instance “a solution comprising A, B and optionally C” may also (essentially) consist of A and B, or (essentially) consist of A, B and C. As used herein, the transitional phrase “consisting essentially of” (and grammatical variants) is to be interpreted as encompassing the recited materials or steps and those that do not materially affect the basic and novel characteristic(s) of the claimed invention. Thus, the term “consisting essentially of” should not be interpreted as equivalent of “comprising”.

Surprisingly, it has been found that a composition comprising
bakuchiol;
an extract from Centella asiatica containing asiaticoside, madecassoside and terminoloside; and
an extract of Prunus amygdalus seeds, exhibits excellent anti-oxidative properties on epidermal skin cells and/or anti-stress properties on stressed skin and can be efficiently used in a number of beneficial skin and hair care cosmetic applications. The cosmetic composition is adapted for topical administration.

The topical cosmetic composition contains bakuchiol. Bakuchiol is a meroterpene phenol abundant in and mainly obtained from the seeds of the Psoralea corylifolia plant. This plant is widely used in Indian as well as in Traditional Chinese medicine to treat a variety of diseases. Preferably, the bakuchiol present in the cosmetic composition according to the present invention is S-bakuchiol (IUPAC name: 4-[(1E,3S)-3-Ethenyl-3,7-dimethyl-1,6-octadien-1-yl]-phenol; CAS Number: 10309-37-2). The bakuchiol may be in a synthetic form or an isolated form. The cosmetic composition contains preferably from 0.1 wt-% to 1 wt-%, more preferably from 0.4 wt-% to 0.6 wt-%, such as 0.5 wt-% bakuchiol, wherein the wt-% are based on the total weight of the cosmetic composition. Bakuchiol is commercially available (e.g., Sytenol®A from SYTHEON LTD).

The topical cosmetic composition further contains an extract from Centella asiatica containing asiaticoside (O-6-deoxy-α-L-mannopyranosyl-(1→4)-O-β-D-glucopyranosyl-(1→6)-β-D-glucopyranosyl(2α,3β,4α)-2,3,23-trihydroxyurs-12-en-28-oate; CAS number: 16830-15-2), madecassoside (O-6-deoxy-α-L-mannopyranosyl-(1->4)-O-β-D-glucopyranosyl-(1->6)-β-D-glucopyranosyl(2α,3β,4α, 6β)-2,3,6,23-tetrahydroxyurs-12-en-28-oate; CAS number: 34540-22-2) and terminoloside (IUPAC name: (2α,3β,4α, 6β)-2,3,6,23-tetrahydroxy-olean-12-en-28-oic acid, O-6-deoxy-α-L-mannopyranosyl-(1→4)-O-β-D-glucopyranosyl-(1→6)-β-D-glucopyranosyl ester; CAS number: 125265-68-1). Asiaticoside, madecassoside and terminoloside are water soluble heterosides.

Preferably, the extract from Centella asiatica is present in the cosmetic composition in a total amount from 0.1 wt-% to 1 wt-% based on the total weight of the cosmetic composition. In a preferred embodiment the extract from Centella asiatica contains
madecassoside, terminoloside and asiaticoside in a total amount of 70.0-90.0 wt-% as determined by HPLC based on the total weight of the extract, and/or
madecassoside and terminoloside in a total amount of 50.0-70.0 wt-% as determined by HPLC based on the total weight of the extract; and/or
asiaticoside in an amount of 10.0-20.0 wt-% based on the total weight of the extract as determined by HPLC.

In a more preferred embodiment the extract from Centella asiatica contains
madecassoside, terminoloside and asiaticoside in a total amount of 70.0-90.0 wt-% as determined by HPLC based on the total weight of the extract; and
madecassoside and terminoloside in a total amount of 50.0-70.0 wt-% as determined by HPLC based on the total weight of the extract; and
asiaticoside in an amount of 10.0-20.0 wt-% based on the total weight of the extract as determined by HPLC. An example of such commercially available extract is CENTEROX™ (Givaudan).

The cosmetic composition claimed and described herein further contains an extract from Prunus amygdalus seeds, preferably in an amount from 3 wt-% to 10 wt-%, wherein the wt-% are based on the total weight of the cosmetic composition. The extract of Prunus amygdalus seeds improves the skin condition through its hydrating and emollient properties. Preferably, the extract of Prunus amygdalus seeds is an extract of Prunus amygdalus dulcis (sweet almond) seeds. Preferably the extract of Prunus amygdalus (dulcis) seeds is a (sweet) almond milk. Such extracts are either commercially available, or can be prepared e.g., by drying the Prunus amygdalus dulcis seeds, if necessary comminuting said seeds, extracting with water and ethanol and filtering. Following distillation to remove the ethanol, the concentrated extract may be dispersed e.g., in water and clear filtrated after an individual ripening time. Suitable commercially available extracts are: Akomilk® Almond (Supplier: Akkot), Almond (sweet) Extract (Supplier: Carruba), Sweet almond milk-NAT (Supplier: Provital) and Almond Extract-NS (Supplier: The Garden of Naturalsolution).

The hair and/or skin cosmetic composition may further comprise one or more cosmetic excipients. Cosmetic preparations typically comprise excipients. Such excipients are known per se and the skilled person is in a position to select them in view of the intended form of application for the mixture of ingredients described herein. Preferably, said excipients are selected from the group consisting of humectants, emollients, moisturizers, thickeners, emulsifiers, colourants, surfactants, preservatives, antioxidants, pH adjusting agents, matifying agents, exfoliating agents, perfumes, essential oils, vitamins, UV filters, and skin conditionings. The cosmetic composition contains a total amount of excipients of up to 96.8 wt-% based on the total weight of the composition.

Cosmetic compositions may be provided in any conventional form known to the skilled person.

In one embodiment, the cosmetic preparation is in the form of a liquid formulation. Such liquid formulations include gels, lotions, milks, emulsions, foams, spray and oils.

In one further embodiment, the cosmetic composition is in the form as a semisolid formulation or as a solid formulation. Such formulations include creams, ointments, powders and sticks.

These types of formulations are known per se.

A preferred embodiment according to the present invention is directed to a cosmetic composition containing:
from 0.1 wt-% to 1 wt-% of bakuchiol;
from 0.1 wt-% to 1 wt-% of the herein described extract from Centella asiatica containing asiaticoside, madecassoside and terminoloside; and
from 3 wt-% to 10 wt-% of extract from Prunus amygdalus seeds, preferably almond milk, with the wt-% being based on the total weight of the composition.

In one embodiment the cosmetic composition is a skin care cosmetic composition. In another embodiment, the cosmetic composition is a hair care cosmetic composition. In a further embodiment, the cosmetic composition is a skin and hair care cosmetic composition.

A further aspect according to the present invention is directed to a method for cosmetic skin treatment wherein the method comprises applying to a skin in need thereof the cosmetic composition according to the present invention.

In the method claimed and described herein, the cosmetic skin treatment preferably comprises
improving the skin condition;
improving the skin elasticity;
increasing the firmness of the skin;
reducing the appearance of uneven pigmentation and texture;
hydrating the skin;
protecting against UV rays and/or radical species;
preventing or reducing the appearance of wrinkles and fine lines on skin; and/or
soothing the skin, including the scalp.

In a preferred embodiment, the cosmetic skin treatment comprises protection against UV rays and/or radical species.

A further aspect according to the present invention is directed to a method for cosmetic hair treatment wherein the method comprises applying to hair in need thereof the cosmetic composition according to the present invention.

Preferably the cosmetic hair treatment comprises:
preventing or reducing the hair loss;
strengthening hair fibres; and/or
reducing and/or delaying the grey colouring of the hair.

To further illustrate the invention, the following examples are provided. These examples are provided with no intent to limit the scope of the invention.

### A. Preparation of a Cosmetic Composition According to the Invention.

A cosmetic composition in the form of a cream for the body and/or the face may, for example, have a composition by weight, as indicated below:

| Ingredient | Supplier | Wt-% |
|---|---|---|
| CENTEROX ™ ¹⁾ | Givaudan | 0.1-1 |
| Bakuchiol | SYTHEON | 0.1-1 |
| Sweet almond milk-NAT | Provital | 3-10 |
| Humectants | | 5-15 |
| Thickeners | | 0.5-2 |
| Colorants | | Up to 2 |
| Emollients | | 5 to 15 |
| Preservatives | | Up to 2 |
| Emulsifiers | | Up to 3 |
| Water | | to 100 |

¹⁾ extract from Centella asiatica containing madecassoside, terminoloside and asiaticoside. The content of HPLC content of heterosides as sum of madecassoside, terminoloside and asiaticoside: 70.0-90.0%. The HPLC content of madecassoside and terminoloside: 50.0-70.0%%. The HPLC content of asiaticoside: 10.0-20.0%.

The process for preparing the skin for the cream for the body and/or the face consists essentially in:

In a container equipped with turbine, blades and vacuum pump, the sweet almond milk, the humectants, and the thickeners were mixed and homogenized under vacuum to furnish the phase A. Subsequently, the phase A is heated to 70 °C.

The colorants and the emollients were added in a lateral vessel and heated at a temperature of 70-75 °C to furnish phase B.

Afterwards, phase B was slowly added to phase A by homogenizing at maximum speed under high vacuum for at least 10-15′. The obtained product was cooled down to 40 °C by homogenizing with turbine and blades under vacuum.

Subsequently phase C (pre-mixed mixture of bakuchiol and preservatives) was added to said product by homogenizing for 5-10′, followed by a phase D (a solution of Centerox in a mixture of water and humectant) which was stepwise added by further homogenizing under vacuum after each addition. The obtained product was cooled down to 25 °C by mixing.

### B. Evaluation of the Protective Effect Against UVA Radiation in Human Keratinocytes.

The production of ROS (reactive oxygen species) by mammalian mitochondria is important because it underlies oxidative damage in many pathologies and contributes to retrograde redox signalling from the organelle to the cytosol and nucleus. Mitochondria constitute the main source of ROS. They convert energy produced from cellular metabolism into a usable form, the adenosine triphosphate (ATP). The process of ATP production involves the transport of hydrogen ions across the mitochondrial membrane, by means of an electron transport chain. In the electron transport chain, electrons are passed through a series of proteins via oxidation-reduction reactions. The last destination of an electron is an oxygen molecule. In normal conditions, the oxygen is reduced to produce water. Nevertheless, in a very low percentage of cases, the electrons passing g through the chain can prematurely and incompletely reduce the oxygen molecule to generate the superoxide radical (O²⁻) and other ROS.

The role of free radicals in tissue damage, due to oxygen deprivation, has been the subject of several studies. Superoxide can inactivate specific enzymes, thus activating the pathway of peroxidation. Under specific stress conditions, this last reaction is predominant, and the cell undergoes through the process of apoptosis or programmed cell death. Heat shock is known to induce oxidative stress in a process linked to the dysfunction of the mitochondrial electron transport chain. The main source of endogenous radicals is represented from mitochondria, plasmatic membrane, endoplasmic reticulum and peroxisome. Nevertheless, several exogenous stimuli, as ionizing radiation, UV light, smoke, inflammatory processes and some human diseases, cause free radicals' production, as well. Particularly, depending on the severity of the damages provoked on mitochondria membranes, the response triggered is different. In the case of leak damages, the electronic chain in the mitochondria membranes would result disturbed, reducing ATP synthesis; thus, depriving the cell of energy. In the other cases of severe damage, the mitochondria membranes and the oxidizing enzymes will be affected, provoking the release of free radicals (ROS).

Approximately, 6% of the solar spectrum is UV radiation (200-400 nm), 52% is visible light (400-760 nm) and the remaining 42% is infrared radiation (760 nm-106 nm). These proportions highlight the importance light-induced damage, rather than historical UVA and UVB associated pathological and dermatological damages.

Although the mechanisms are not fully understood, it appears that UVA radiation is involved in human photoaging. For these reasons, in this study, this experiment aims at evaluating the protective effect against oxidative damage of a cosmetic composition according to the invention in human keratinocytes.

### B.1 Materials

| INGREDIENTS | Provider | | Abbreviation |
|---|---|---|---|
| SYTENOL ® A | SYTHEON LTD | Extract from | SYTENOL A |
| | | Babchi seeds | |
| | | containing at | |
| | | least 95 wt-% | |
| | | bakuchiol | |
| Sweet almond | Provital | | SWEET ALMOND |
| milk-NAT | | | |
| CENTEROX ™ ¹⁾ | Givaudan | Extract from | CENTEROX |
| | | Centella | |
| | | asiatica | |
| | | containing | |
| | | madecassoside, | |
| | | terminoloside | |
| | | and | |
| | | asiaticoside. | |

¹⁾ The HPLC content of heterosides as sum of madecassoside, terminoloside and asiaticoside: 70.0-90.0%. The HPLC content of madecassoside and terminoloside: 50.0-70.0%. The HPLC content of asiaticoside: 10.0-20.0%.

The experiment was conducted using human keratinocytes cell line (HaCat).

### B.2 Material and Methods

### B.2.1 Analytical Equipment

Stereoscopic microscope, cell culture incubator (37 °C, 5% CO2, 90% relative humidity (RH), statistical analysis software, laminar flow hood, micropipettes, pipettes, propipette, rack, Bürker chamber, freezer −20 °C, freezer −80 °C, rack, heating block, vortex, Luzchem Irradiator LZC-420 with UVA lamps, Glomax Discovery Promega Multi-mode detection system (Promega), plate reader spectrophotometer and consumables.

### B.2.2 Reagents

DMEM low glucose medium (Gibco), FBS (Gibco), Penicillin/Streptomycin (Life Technologies), LGlutamine (Gibco), Phosphate buffered saline (Gibco), Trypan Blue Solution (Bio-Rad), (Gibco), MTT reagent [3-(4,5-Dimethylthiazol-2-yl)-TrypsinEDTA 2,5-Diphenyltetrazolium Bromide] (Invitrogen), dimethyl sulfoxide (DMSO, Sigma-Aldrich) and Intracellular ROS detection kit (Sigma Aldrich), Vitamine C (Merck).

### B.2.3 Procedure

### B.2.3.1 Cell Viability (MTT Assay)

Cell numbers and viability were determined using Trypan-Blue staining and counting in a Bürker chamber under the microscope. For the MTT viability assay, human keratinocytes were cultured overnight at a 10.000 cells/well density in a 96 well plate, in supplemented growth medium. 24 h later, the culture medium was replaced with fresh medium containing the tested products (SYTENOL A, SWEET ALMOND, CENTEROX and mixtures of SYTENOL A, SWEET ALMOND and CENTEROX) 00 at 8 different concentrations. After 24 h of incubation, the medium was removed, and MTT solution was added to each well. Plates were incubated at 37 °C for 3 h. MTT reagent was removed and DMSO at 100% was added to each well to solubilize formazan crystals, then the absorbance was measured at 550 nm and 620 nm as a reference on a scanning multi-well spectrophotometer.

### B.2.3.2 ROS Quantification

Human keratinocytes were cultured overnight at a 10.000 cells/well density in a black 96-well plate, in growth media. 24 h later, the culture media was removed and replaced by new culture medium supplied with the tested products at the selected concentrations or vitamine C at 0.0035%. After additional 24 h of incubation, PBS and ROS master mix (Intracellular ROS detection kit (Sigma Aldrich)) were added in all cultured wells. 2 hours after ROS master mix addition to cells, ROS levels were measured in all samples. The intracellular ROS react with a fluorogenic sensor localized in the cytoplasm, resulting in a fluorescent product whose appearance is proportional to ROS levels. Fluorescence quantification was measured at λex=490/λem=525. In parallel, cell viability was quantified through MTT assay to normalize ROS levels to the number of live cells due to UVA-induced cytotoxicity.

### B.2.4 Statistical Analysis

The MTT assay was set with 8 replicates per condition and 16 for control, and ROS quantification assay was set with 5 replicates per condition. Data outliers were identified with ROUT method (Q=5%) and excluded from the analysis if found. Raw data were statistically analyzed by Unpaired t-test or ordinary one-way ANOVA test and Dunnet's post hoc multiple comparisons test.

Statistical significance was set at p<0.05, 95% of confidence. Bars in the charts represent the mean value for each condition and error bars indicate the standard error of the mean (SEM) for each group of values.

### B.3 Results

### B.3.1 Cell Viability (MTT Assay)

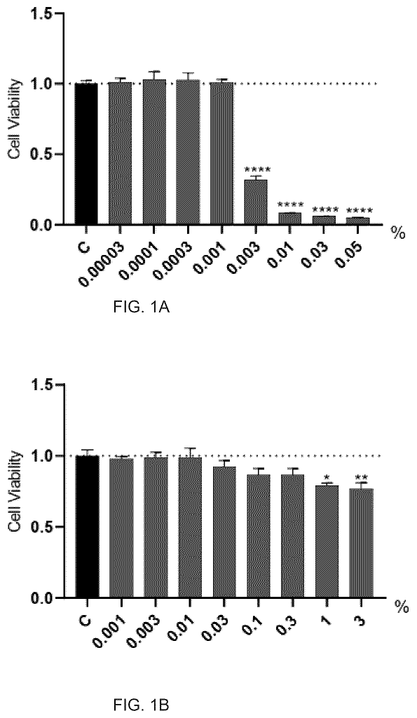

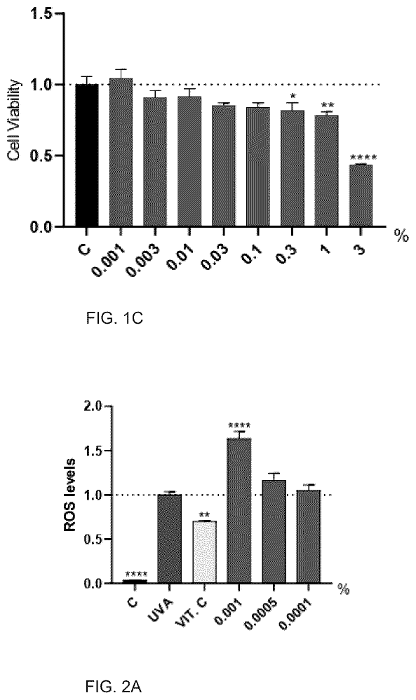

The effect of SYTHENOL A, SWEET ALMOND and CENTEROX on cell viability was tested. The results are summarized in FIG. 1. As shown by FIG. 1:
Treatment with SYNTENOL A did not affect cell viability when used at concentrations below 0.003 wt-%;
Treatment with SWEET ALMOND did not affect cell viability when used at concentrations below 1 wt-%;
Treatment with CENTEROX did not affect cell viability when used at concentrations below 0.3 wt-%.

### B.3.2 ROS Quantification

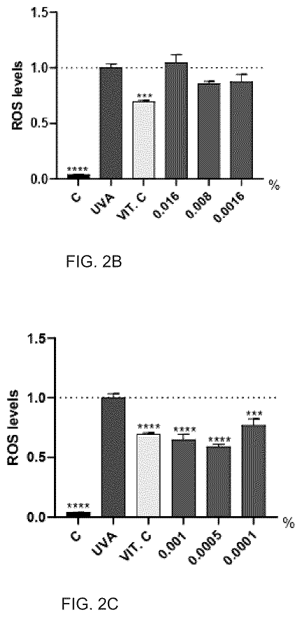

The phototoxic effect of SYTHENOL A, SWEET ALMOND and CENTEROX was tested. The results are summarized in FIG. 2. As shown by Figure
UVA exposure increased ROS levels by 2495±89% compared to the non-treated control;
Treatment with the antioxidant control vitamin C decreased the ROS levels by 30.3±2.9% compared to the UVA-irradiated control;
SYTHENOL A and Sweet Almond exhibit a phototoxic effect.
Treatment with CENTEROX reduced UVA-induced ROS levels by 35.1±4.9%, 41.0±4.9% and 23.2±4.9% when used at a concentration of 0.001 wt-g, 0.0005 wt-g and 0.0001 wt-g, respectively.

### B.3.3 Cell Viability (MTT Assay)

The following two compositions were prepared by mixing Sweet almond, CENTEROX and SYTHENOL A in fresh medium:
1^{st} composition contains 0.008 wt-% Sweet almond, 0.0005 wt-% CENTEROX and 0.0005 wt-% SYTHENOL A
2^{nd} composition contains 0.0016 wt-% Sweet almond, 0.0001 wt-% CENTEROX and 0.0001 wt-% SYTHENOL A.

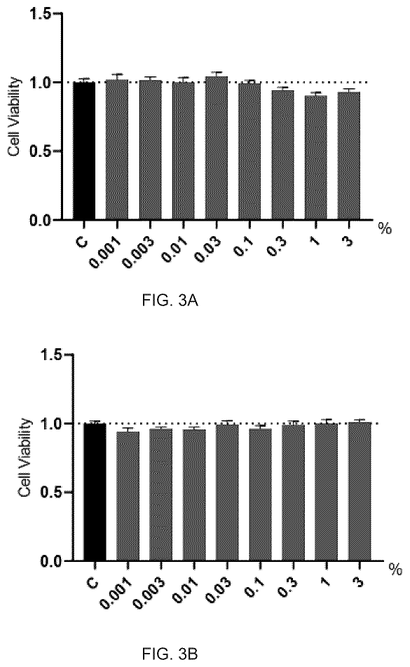

The effect of the 1^{st} and 2^{nd} compositions on cell viability was tested. The results are summarized in FIG. 3. As shown by FIG. 3, none of the compositions affected cell viability at any of the tested concentrations.

### B.3.4 ROS Quantification

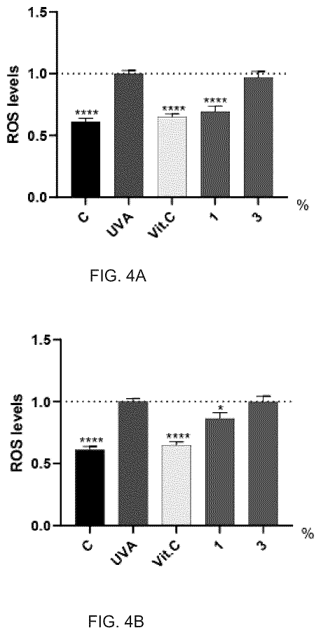

The phototoxic effect of the 1^{st} composition and the 2^{nd} composition described under section B.3.4 was tested. The results are summarized in FIG. 4. As shown by FIG. 4
UVA exposure increased ROS levels by 63.9±6.3% compared to the non-treated control.
Treatment with the antioxidant control vitamin C (Vit C) decreased the ROS levels by 35.1±3.8% compared to the UVA-irradiated control.
Treatment with the 1^{st} composition reduced the levels of UVA-induced ROS levels by 30.7±5.1% when used at a concentration of 1%.
Treatment with the 2^{nd} composition reduced the levels of UVA-induced ROS levels by 13.9±5.2% when used at a concentration of 1%.

The results depicted in FIG. 2 show that when used alone only CENTEROX exhibits a significant protective effect against UVA-induced damages at concentrations of 0.001 wt-%, and 0.0005 wt-%. The results depicted in FIG. 4 shows that the combination of SYTHENOL A, Sweet Almond and CENTEROX exhibits a protective effect against the damages induced by UVA comparative to the protective effect exhibited by vitamin C, but at a concentration of each of the three components (SYTHENOL A, Sweet Almond and CENTEROX) 100 times lower than the concentration tested at section B. 3.2 (FIG. 2).

1. A cosmetic composition for hair and/or skin care adapted for topical application, wherein said cosmetic composition contains:
bakuchiol;
an extract from Centella asiatica containing asiaticoside, madecassoside and terminoloside; and
an extract from Prunus amygdalus seeds.

2. The cosmetic composition according to claim 1, wherein the bakuchiol is S-bakuchiol.

3. The cosmetic composition according to claim 1, wherein the bakuchiol is present in an amount from 0.1 wt-% to 1 wt-%, wherein the wt-% are based on the total weight of the cosmetic composition.

4. The cosmetic composition according to claim 1, wherein the extract from Centella asiatica contains asiaticoside, madecassoside and terminoloside in a total amount of 70.0-90.0 wt-% based on the total weight of the extract.

5. The cosmetic composition according to claim 1, wherein the extract from Centella asiatica is present in an amount from 0.1 wt-% to 1 wt-%, wherein the wt-% are based on the total weight of the cosmetic composition.

6. The cosmetic composition according to claim 4, wherein the extract from Centella asiatica is present in an amount from 0.1 wt-% to 1 wt-%, wherein the wt-% are based on the total weight of the cosmetic composition.

7. The cosmetic composition according to claim 1, wherein the extract of Prunus amygdalus seeds is present in an amount from 3 wt-% to 10 wt-%, wherein the wt-% are based on the total weight of the cosmetic composition.

8. The cosmetic composition according to claim 1, wherein the Prunus amygdalus is Prunus amygdalus dulcis.

9. The cosmetic composition according to claim 1, wherein said cosmetic composition contains:
from 0.1 wt-% to 1 wt-% of bakuchiol;
from 0.1 wt-% to 1 wt-% of the extract from Centella asiatica; and
from 3 wt-% to 10 wt-% of the extract from Prunus amygdalus seeds, with the wt-% being based on the total weight of the composition.

10. The cosmetic composition according to claim 4, wherein said cosmetic composition contains:
from 0.1 wt-% to 1 wt-% of bakuchiol;
from 0.1 wt-% to 1 wt-% of the extract from Centella asiatica; and
from 3 wt-% to 10 wt-% of the extract from Prunus amygdalus seeds, with the wt-% being based on the total weight of the composition.

11. The cosmetic composition according to claim 1, further containing one or more cosmetic excipients selected from the group consisting of humectants, emollients, moisturizers, thickeners, emulsifiers, colourants, surfactants, preservatives, antioxidants, pH adjusting agents, matifying agents, exfoliating agents, perfumes, essential oils, vitamins, UV filters, and skin conditionings.

12. The cosmetic composition according to claim 1, wherein said cosmetic composition is formulated as
a liquid formulation, preferably a gel, a lotion, a milk, an emulsion, a foam, a spray, or an oil; or
a semisolid or solid formulation, preferably a cream, an ointment, a stick, or a powder.

13. A method for cosmetic skin treatment wherein the method comprises applying to a skin in need thereof the cosmetic composition according to claim 1.

14. The method according to claim 13, wherein the cosmetic skin treatment comprises
improving the skin condition;
improving the skin elasticity;
increasing the firmness of the skin;
reducing the appearance of uneven pigmentation and texture;
hydrating the skin;
protecting against UV rays and/or radical species;
preventing or reducing the appearance of wrinkles and fine lines on skin; and/or
soothing the skin, including the scalp.

15. A method for cosmetic hair treatment wherein the method comprises applying to hair in need thereof the cosmetic composition according to claim 1.

16. The method according to claim 15, wherein the cosmetic hair treatment comprises:
preventing or reducing the hair loss;
strengthening hair fibers; and/or
reducing and/or delaying the grey colouring of the hair.
